(19) United States
(12) Patent Application Publication
(10) Pub. No.: US 2025/0173923 A1
(43) Pub. Date: May 29, 2025

(54) IMAGE PROCESSING DEVICE, IMAGE PROCESSING METHOD, AND STORAGE MEDIUM

(71) Applicant: NEC Corporation, Minato-ku, Tokyo (JP)

(72) Inventor: Ryosaku SHINO, Tokyo (JP)

(73) Assignee: NEC Corporation, Minato-ku, Tokyo (JP)

(21) Appl. No.: 18/729,113

(22) PCT Filed: Feb. 1, 2022
(86) PCT No.: PCT/JP2022/003805

§ 371 Date: Jul. 15, 2024

(51) Int. Cl. G06T 11/00; G06T 11/60

(52) U.S. Cl. CPC G06T 11/003; G06T 11/60

(57) ABSTRACT

The image processing device 1X mainly includes a three-dimensional reconstruction means 31X, a matching means 32X, a complement means 33X, and a display control means 34X. The three-dimensional reconstruction means generates, based on endoscopic images of an examination target captured by a photographing unit provided in an endoscope, reconstruction data obtained by three-dimensionally reconstructing the examination target. The matching means performs matching between a three-dimensional model of the examination target and the reconstruction data. The complement means generates, based on a result of the matching, complemented reconstruction data which is the reconstruction data complemented with the three-dimensional model. The display control means displays the complemented reconstruction data on a display device.

### TECHNICAL FIELD

The present disclosure relates to a technical field of an image processing device, an image processing method, and a storage medium for processing an image acquired in endoscopic examination.

### BACKGROUND

An endoscopic examination system for displaying images taken in the lumen of an organ is known. For example, Patent Literature 1 discloses a technique for generating, on the basis of images taken by endoscope, three-dimensional model data of the examination target to thereby display a three-dimensional model image. Patent Literature 2 discloses a technique for generating volume data (volumetric image) representing a large bowel by capturing a three-dimensional region in which the large bowel is included by an X-ray CT device. In addition, Non-Patent Literature 1 discloses a technique for reconstructing the three-dimensional shape of a stomach from captured images using the SfM (Structure from Motion) method. Furthermore, Non-Patent Literature 2 discloses a non-rigid alignment (registration) method of three-dimensional shapes.

### CITATION LIST

### Patent Literature

Patent Literature 1: WO2017/203814

Patent Literature 2: JP2011-139797A

### Non-Patent Literature

Non-Patent Literature 1: Aji Resindra Widya et al. “3D Reconstruction of Whole Stomach from Endoscope Video Using Structure-from-Motion”, 2019 41st Annual International Conference of the IEEE Engineering in Medicine and Biology Society, pp. 3900-3904.

Non-Patent Literature 2: Dai, H et al. “Non-rigid 3D Shape Registration using an Adaptive Template”, The European Conference on Computer Vision (ECCV) Workshops, 2018.

### SUMMARY

### Problem to be Solved

When three-dimensional model data of an examination target is generated and displayed as a three-dimensional model image on the basis of images captured by an endoscope, the three-dimensional model image could become incomplete due to the existence of an unphotographed region in the examination target.

In view of the above-described issue, it is therefore an example object of the present disclosure to provide an image processing device, an image processing method, and a storage medium capable of suitably displaying an examination target in an endoscopic examination.

### Means for Solving the Problem

One mode of the image processing device is an image processing device including:
a three-dimensional reconstruction means configured to generate, based on endoscopic images of an examination target captured by a photographing unit provided in an endoscope,
   reconstruction data obtained by three-dimensionally reconstructing the examination target;
a matching means configured to perform matching between a three-dimensional model of the examination target and the reconstruction data;
a complement means configured to generate, based on a result of the matching, complemented reconstruction data which is the reconstruction data complemented with the three-dimensional model; and
a display control means configured to display the complemented reconstruction data on a display device.

One mode of the image processing method is an image processing method executed by a computer, the image processing method including:
generating, based on endoscopic images of an examination target captured by a photographing unit provided in an endoscope,
   reconstruction data obtained by three-dimensionally reconstructing the examination target;
performing matching between a three-dimensional model of the examination target and the reconstruction data;
generating, based on a result of the matching, complemented reconstruction data which is the reconstruction data complemented with the three-dimensional model; and
displaying the complemented reconstruction data on a display device.

One mode of the storage medium is a storage medium storing a program executed by a computer, the program causing the computer to:
generate, based on endoscopic images of an examination target captured by a photographing unit provided in an endoscope,
   reconstruction data obtained by three-dimensionally reconstructing the examination target;
perform matching between a three-dimensional model of the examination target and the reconstruction data;
generate, based on a result of the matching, complemented reconstruction data which is the reconstruction data complemented with the three-dimensional model; and
display the complemented reconstruction data on a display device.

### Effect

An example advantage according to the present invention is to suitably display an examination target in an endoscopic examination.

### BRIEF DESCRIPTION OF THE DRAWINGS

FIG. 1 It illustrates a schematic configuration of an endoscopic examination system.

FIG. 2 It illustrates a hardware configuration of an image processing device.

FIG. 3 It is a functional block diagram of the image processing device.

FIG. 4 It is a diagram showing an outline of a process of generating complemented reconstruction data.

FIG. 5 It is an example of a flowchart showing an outline of a display process performed by the image processing device during the endoscopic examination in the first example embodiment.

FIG. 6 It illustrates a first display example of the examiner confirmation screen image.

FIG. 7 It illustrates a second display example of the examiner confirmation screen image.

FIG. 8 It illustrates a third display example of the examiner confirmation screen image.

FIG. 9 It illustrates a fourth display example of the examiner confirmation screen image.

FIG. 10 It is a block diagram of an image processing device according to the second example embodiment.

FIG. 11 It is an example of a flowchart showing a processing procedure of the image processing device according to the second example embodiment.

### EXAMPLE EMBODIMENTS

Hereinafter, example embodiments of an image processing device, an image processing method, and a storage medium will be described with reference to the drawings.

### First Example Embodiment

### (1) System Configuration

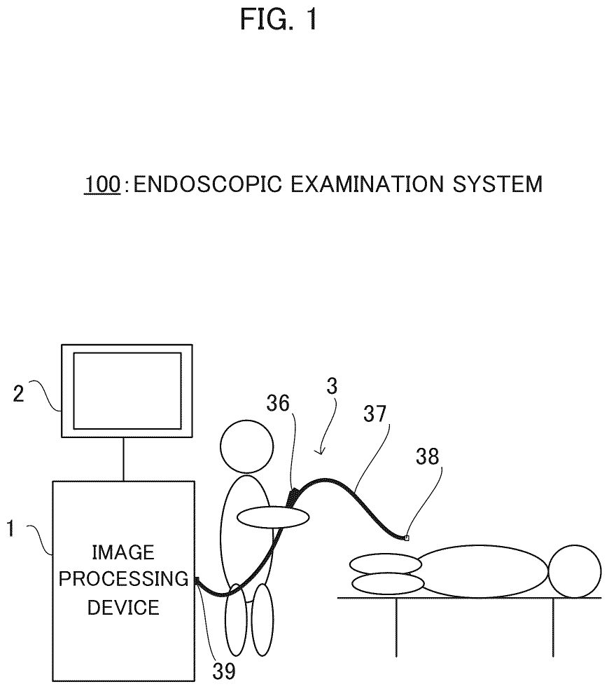

FIG. 1 shows a schematic configuration of an endoscopic examination system 100. During examination (including treatment) using an endoscope, the endoscopic examination system 100 complements three-dimensional data of a target organ of examination with a three-dimensional model, wherein the three-dimensional data is constructed by endoscopic images and the three-dimensional model is generated on the basis of images obtained in a preliminary examination performed prior to the endoscopic examination. Thus, the endoscopic examination system 100 assists an examiner such as a doctor to conduct the endoscopic examination. The preliminary examination is an examination in which scan data of the target organ of examination is generated by a CT or an MRI or the like and a diagnosis based on the generated scan data is made. In addition, in the preliminary examination, the process of generating the scan data of the target organ of examination is required to be performed, and the diagnosis may not necessarily be made.

As shown in FIG. 1, the endoscopic examination system 100 mainly includes an image processing device 1, a display device 2, and an endoscope 3 connected to the image processing device 1.

The image processing device 1 acquires images (also referred to as “endoscopic images Ic”) captured by the endoscope 3 in time series from the endoscope 3 and displays a screen image (also referred to as “examiner confirmation screen image”) for the examiner to check the endoscope on the display device 2. The endoscopic images Ic are images taken at predetermined time intervals in at least one of the insertion process of the endoscope 3 to a subject and/or the ejection process of the endoscope 3 from the subject. In the present example embodiment, the image processing device 1 generates data (also referred to as “reconstruction data Mr”) obtained by reconstructing a three-dimensional shape of the organ (digester) of examination of the subject using the endoscopic images Ic, and performs matching (alignment) thereof with a three-dimensional model (also referred to as “preliminary examination model Mp”) of the target organ of examination of the subject, wherein the preliminary examination model Mp is generated based on a result of the preliminary examination using a CT or an MRI. Then, the image processing device 1 generates reconstruction data Mr (also referred to as “complemented reconstruction data Mrc”) complemented to represent the whole target organ of examination by complementing the reconstruction data Mr with the preliminary examination model Mp based on the matching (alignment) result. Then, the image processing device 1 causes the display device 2 to display an image representing the complemented reconstruction data Mrc. The image processing device 1 may generate and display the complemented reconstruction data Mrc during the endoscopic examination using the endoscopic images obtained during the endoscopic examination, or may generate and display the complemented reconstruction data Mrc after the endoscopic examination.

The display device 2 is a display or the like for displaying information based on the display signal supplied from the image processing device 1.

The endoscope 3 mainly includes an operation unit 36 for examiner to perform a predetermined input, a shaft 37 which has flexibility and which is inserted into the organ to be photographed of the subject, a tip unit 38 having a built-in photographing unit such as an ultra-small image pickup device, and a connecting unit 39 for connecting with the image processing device 1.

In the following description, an explanation will be given on the assumption that a stomach is mainly targeted in the endoscopic examination, however examples of the examination target include not only the stomach but also any other digestive tract (digestive organ) such as a large bowel, an esophageal, a small bowel and a duodenum. Examples of the endoscope in the present disclosure include a laryngendoscope, a bronchoscope, an upper digestive tube endoscope, a duodenum endoscope, a small bowel endoscope, a large bowel endoscope, a capsule endoscope, a thoracoscope, a laparoscope, a cystoscope, a cholangioscope, an arthroscope, a spinal endoscope, a blood vessel endoscope, and an epidural endoscope.

Further, in the following, the term “part of interest” indicates any part of the examination target that the examiner needs to pay attention to in the endoscopic examination. Examples of the part of interest include a lesion part, an inflammation part, a point with an operating mark or other cuts, a point with a fold or a protrusion, a point on the wall surface of the lumen where the tip unit 38 of the endoscope 3 tends to get contact (caught). The conditions of the lesion part to be detected in endoscopic examination are exemplified as (a) to (f) below.
(a) Head and neck: pharyngeal cancer, malignant lymphoma, papilloma
(b) Esophagus: esophageal cancer, esophagitis, esophageal hiatal hernia, Barrett's esophagus, esophageal varices, esophageal achalasia, esophageal submucosal tumor, esophageal benign tumor
(c) Stomach: gastric cancer, gastritis, gastric ulcer, gastric polyp, gastric tumor
(d) Duodenum: duodenal cancer, duodenal ulcer, duodenitis, duodenal tumor, duodenal lymphoma
(e) Small bowel: small bowel cancer, small bowel neoplastic disease, small bowel inflammatory disease, small bowel vascular disease
(f) Large bowel: colorectal cancer, colorectal neoplastic disease, colorectal inflammatory disease; colorectal polyps, colorectal polyposis, Crohn's disease, colitis, intestinal tuberculosis, hemorrhoids.

### (2) Hardware Configuration

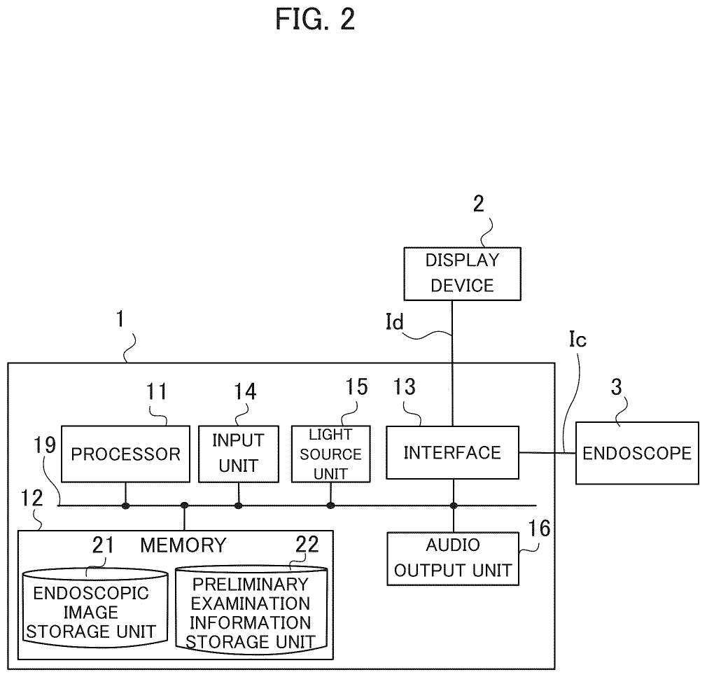

FIG. 2 shows the hardware configuration of the image processing device 1. The image processing device 1 mainly includes a processor 11, a memory 12, an interface 13, an input unit 14, a light source unit 15, and an audio output unit 16. Each of these elements is connected to each other via a data bus 19.

The processor 11 executes a predetermined process by executing a program or the like stored in the memory 12. The processor 11 is one or more processors such as a CPU (Central Processing Unit), a GPU (Graphics Processing Unit), and a TPU (Tensor Processing Unit). The processor 11 may be configured by plural processors. The processor 11 is an example of a computer.

The memory 12 is configured by a variety of volatile memories which is used as working memories, and nonvolatile memories which stores information necessary for the process to be executed by the image processing device 1, such as a RAM (Random Access Memory) and a ROM (Read Only Memory). The memory 12 may include an external storage device such as a hard disk connected to or built in to the image processing device 1, or may include a storage medium such as a removable flash memory. The memory 12 stores a program for the image processing device 1 to execute each process in the present example embodiment.

The memory 12 functionally includes an endoscopic image storage unit 21 and a preliminary examination information storage unit 22.

The endoscopic image storage unit 21 stores a series of endoscopic images Ic taken by the endoscope 3 in the endoscopic examination based under the control of the processor 11. These endoscopic images Ic are images used for generating the reconstruction data Mr. For example, the endoscopic images Ic are stored in the endoscopic image storage unit 21 in association with the identification information (e.g., patient ID) of the subject and time stamp information.

The preliminary examination information storage unit 22 stores the preliminary examination information that is information regarding the examination result of the preliminary examination of the subject using a CT device or an MRI device or the like. The preliminary examination information includes: scan data (also referred to as “preliminary scan data”) of the target organ of examination of the subject using a device such as a CT device and an MRI device; a preliminary examination model Mp that is a three-dimensional shape model of the target organ of the examination generated from the preliminary scan data; and meta data associated with the preliminary scan data and the preliminary examination model Mp. The metadata does not necessarily need to be stored in the preliminary examination information storage unit 22.

The preliminary examination model Mp is generated by extracting a three-dimensional shape of the target organ of the examination from preliminary scan data such as three-dimensional CT images and MRI data. For example, the preliminary examination model Mp is herein represented in a predetermined three-dimensional coordinate system. The preliminary examination information storage unit 22 may further include coordinate transformation information between the three-dimensional coordinate system of the preliminary examination model Mp and the coordinate system (two-dimensional or three-dimensional coordinate system) of the preliminary scan data. This coordinate transformation information is generated in the process of generating a preliminary examination model Mp from the preliminary scan data. The process of generating the preliminary examination model Mp from the preliminary scan data may be performed in advance by the image processing device 1 before the endoscopic examination, or may be performed by a device other than the image processing device 1 before the endoscopic examination.

For example, the above-mentioned meta data is data which is attached to the preliminary scan data through an annotation work by a doctor in charge of the preliminary examination, or, data obtained by applying a CAD (Computer Aided Diagnosis) to the preliminary scan data. For example, the above-described annotation work is a work to be conducted by a doctor in charge of the preliminary examination as to specifying the part of interest of preliminary scan data with reference to the displayed preliminary scan data and inputting a comment or the like regarding the specified part of interest of the preliminary scan data to a computer. Then, for example, the meta data includes information regarding the part of interest such as a lesion part detected in the preliminary examination. For example, the meta data includes position information (e.g., a coordinate value in a coordinate system used in the preliminary scan data), which indicates the part of interest to be noticed in the endoscopic examination, and content information, which represents a diagnosis result regarding the position (that is, the part of interest) specified by the position information or the type of the part of interest. The meta data may also include information regarding the attributes of one or more doctors in charge of the preliminary examination (including the name of the doctor in charge and the affiliation information thereof), as will be described later.

Here, at least either the endoscopic image storage unit 21 or the preliminary examination information storage unit 22 may be provided in, instead of the memory 12, an external device capable of wired or wireless data communication with the image processing device 1. In this case, the external device may be one or more server devices capable of data communication with the image processing device 1 via a communication network.

In addition to the above-described information, the memory 12 may store various kinds of information necessary for processing in the present example embodiment. For example, when the image processing device 1 performs a CAD based on the endoscopic images Ic, the memories 12 may further store parameters and the like regarding a lesion detection model required to perform the CAD. In this case, the lesion detection model is, for example, a machine learning model such as a neural network and a support vector machine, and is configured to output, when an endoscopic image Ic is inputted thereto, the presence or absence of a lesion part in the inputted endoscopic image Ic and, if there is a lesion part, its position information (which may be region information) in the inputted endoscopic image Ic. In the case where the lesion detection model is configured by a neural network, the memory 12 stores various parameters such as, for example, a layer structure, a neuron structure of each layer, the number of filters and the size of filters in each layer, and the weight for each element of each filter.

The interface 13 performs an interface operation between the image processing device 1 and an external device. For example, the interface 13 supplies the display information “Id” generated by the processor 11 to the display device 2. Further, the interface 13 supplies the light generated by the light source unit 15 to the endoscope 3. The interface 13 also provides an electrical signal to the processor 11 indicative of the endoscopic image Ic supplied from the endoscope 3. The interface 13 may be a communication interface, such as a network adapter, for wired or wireless communication with the external device, or a hardware interface compliant with a USB (Universal Serial Bus), a SATA (Serial AT Attachment), or the like.

The input unit 14 generates an input signal based on the operation by the examiner. Examples of the input unit 14 include a button, a touch panel, a remote controller, and a voice input device. The light source unit 15 generates light for supplying to the tip unit 38 of the endoscope 3. The light source unit 15 may also incorporate a pump or the like for delivering water and air to be supplied to the endoscope 3. The audio output unit 16 outputs a sound under the control of the processor 11.

### (3) Functional Blocks

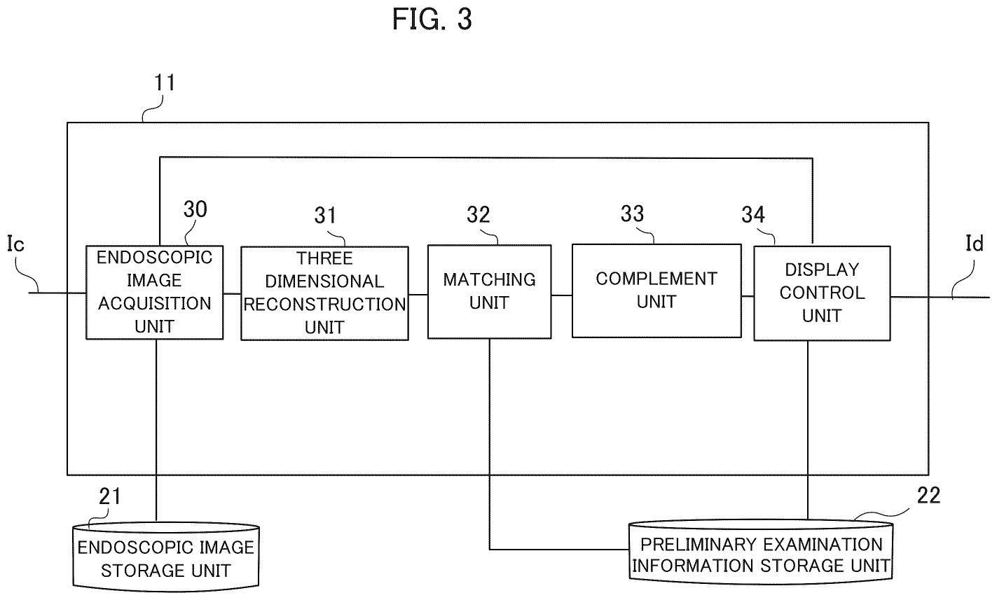

FIG. 3 is a functional block diagram of the image processing device 1. As shown in FIG. 3, the processor 11 of the image processing device 1 functionally includes an endoscopic image acquisition unit 30, a three-dimensional reconstruction unit 31, a matching unit 32, a complement unit 33, and a display control unit 34. In FIG. 3, blocks to exchange data with each other are connected by a solid line, but the combination of the blocks to exchange data with each other is not limited to FIG. 3. The same applies to the drawings of other functional blocks described below.

The endoscopic image acquisition unit 30 acquires the endoscopic image Ic taken by the endoscope 3 through the interface 13 at predetermined intervals. Then, the endoscopic image acquisition unit 30 supplies the acquired endoscopic image Ic to the three-dimensional reconstruction unit 31. In addition, the endoscopic image acquisition unit 30 stores the acquired endoscopic image Ic in the endoscopic image storage unit 21 in association with the time stamp, patient ID, and/or the like. The endoscopic image acquisition unit 30 also supplies the acquired most recent endoscopic image Ic to the display control unit 34.

The three-dimensional reconstruction unit 31 generates reconstruction data Mr, which indicates the three-dimensional shape of the photographed organ, based on a plurality of endoscopic images Ic obtained by the endoscopic image acquisition unit 30 during the endoscopic examination. Examples of the reconstruction data Mr include point cloud data having three-dimensional position information.

In this instance, for example, upon acquiring the necessary number of endoscopic images Ic required for generating the reconstruction data Mr, the three-dimensional reconstruction unit 31 configures the reconstruction data Mr using a technique for reconstructing the three-dimensional shape of a subject and the relative position of the photographing unit from a plurality of images. Examples of such a technique include the Structure from Motion (SfM). Thereafter, each time acquiring a predetermined number of endoscopic images Ic, the three-dimensional reconstruction unit 31 updates the reconstruction data Mr. The predetermined number may be one or more and is predetermined to a value in consideration of the processing capacity of the image processing device 1, for example. The three-dimensional reconstruction unit 31 supplies the generated (or updated) reconstruction data Mr to the matching unit 32. The method of generating the reconstruction data Mr will be described later.

The matching unit 32 performs matching (alignment) between the reconstruction data Mr supplied from the three-dimensional reconstruction unit 31 and the preliminary examination model Mp stored in the preliminary examination information storage unit 22, and supplies the matching (alignment) result to the complement unit 33. In this instance, for example, the matching unit 32 performs non-rigid registration (non-rigid alignment) and generates data representing the reconstruction data Mr and the preliminary examination model Mp after the non-rigid registration in a common three-dimensional coordinate system (also referred to as “common coordinate system”). Then, for example, the matching unit 32 generates the coordinate transformation information regarding the above-described data or/and the common coordinate system as the matching result to be supplied to the complement unit 33. The above-described coordinate transformation information herein includes, for example, coordinate transformation information from the coordinate system adopted in the reconstruction data Mr to the common coordinate system, and coordinate transformation information from the coordinate system adopted in the preliminary examination model Mp to the common coordinate system.

Based on the matching result supplied from the matching unit 32, the complement unit 33 generates complemented reconstruction data Mrc in which the reconstruction data Mr is complemented by the preliminary examination model Mp so as to represent the whole of the target organ of examination (the large bowel, herein), and supplies the generated complemented reconstruction data Mrc to the display control unit 34. In this instance, on the basis of the matching result, the complement unit 33 determines that a region (portion), of the examination target represented by the preliminary examination model Mp, which does not correspond to the reconstruction data Mr is a region (also referred to as “non-photographed region”) which is not photographed by the endoscope. Then, the complement unit 33 generates the complemented reconstruction data Mrc obtained by adding data of the preliminary examination model Mp corresponding to the non-photographed region to the reconstruction data Mr. It is noted that the non-photographed region occurs even in a region of the examination target within the photographed range by the endoscope 3 because the endoscopic images Ic that correctly represent the state of the examination target were not generated due to the blur or the like. As a result, a hole or the like could be generated in the reconstruction data Mr. Even in such cases, the complement unit 33 generates data (so-called patch) for filling the hole of the examination target generated in the reconstruction data Mr, on the basis of the matching result generated by the matching unit 32 and the preliminary examination model Mp, and generates the complemented reconstruction data Mrc in which the generated data is added to the reconstruction data Mr.

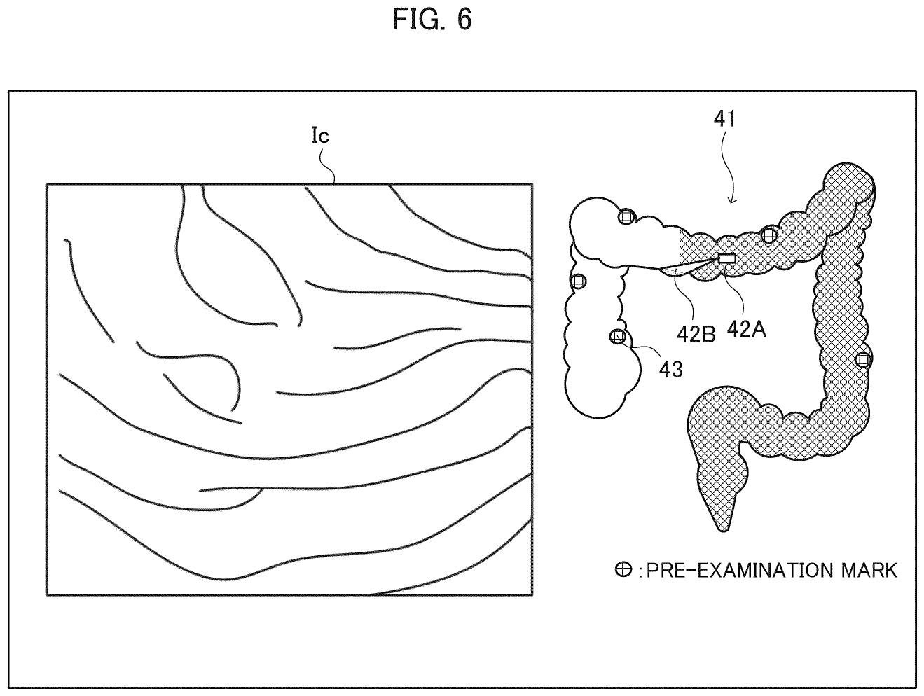

The display control unit 34 generates display information Id of the examiner confirmation screen image, on the basis of the complemented reconstruction data Mrc generated by the complement unit 33, preliminary examination information, and endoscopic images Ic, and supplies the generated display information Id to the display device 2, thereby causing the display device 2 to display the examiner confirmation screen image. In this event, for example, the display control unit 34 arranges and displays the endoscopic image Ic generated in real time and the latest complemented reconstruction data Mrc side by side on the examiner confirmation screen image. Further, for example, the display control unit 34 may display information on the part of interest indicated by the meta data included in the preliminary examination information storage unit 22 on the examiner confirmation screen image in association with the endoscopic image Ic supplied from the endoscopic image acquisition unit 30. In other examples, the display control 34 may output information for guidance or warning regarding the examiner's photographing by the endoscope 3. The information may be outputted on the examiner confirmation screen image or by the audio output unit 16. The display examples of the examiner confirmation screen image will be specifically described with reference to FIGS. 6 to 9.

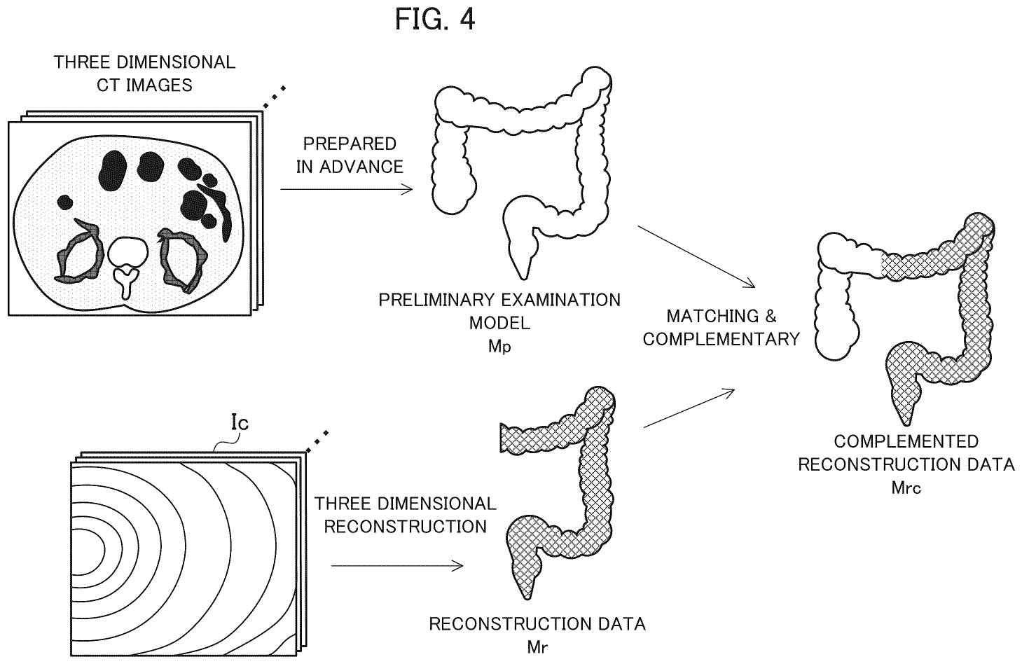

FIG. 4 is a diagram showing an outline of processing performed by the three-dimensional reconstruction unit 31 and the matching unit 32. Although FIG. 4 illustrates the case where a large bowel is examined for convenience of explanation, the outline of the processing shown in FIG. 4 is also applied to a stomach or any other digestive tract.

The three-dimensional reconstruction unit 31 generates reconstruction data Mr corresponding to the three-dimensional shape of the region (already-photographed region) in the digestive tract already photographed by the endoscope 3, on the basis of a plurality of endoscopic images Ic acquired up to the present during the endoscopic examination. In addition, in this case, the preliminary examination model Mp is generated from a plurality of CT images (three-dimensional CT images) of the target organ of examination of the subject.

Then, the matching unit 32 performs matching (non-rigid registration) between the preliminary examination model Mp stored in the preliminary examination information storage unit 22 and the reconstruction data Mr. Thereby, the matching unit 32 associates the preliminary examination model Mp representing the whole organ with the reconstruction data Mr corresponding to the photographed region in the common coordinate system. Then, on the basis of the matching result representing the association (e.g., the coordinate transformation information from the respective data to the common coordinate system), the complement unit 33 generates the complemented reconstruction data Mrc that is the reconstruction data Mr complemented with the preliminary examination model Mp. As a result, complemented reconstruction data Mrc, which is three-dimensional data representing the whole organ (here, the large bowel) subjected to examination including the non-photographed region is obtained.

Each component of the endoscope image acquisition unit 30, the three-dimensional reconstruction unit 31, the matching unit 32, the complement unit 33 and the display control unit 34 can be realized, for example, by the processor 11 which executes a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

### (4) Processing Flow

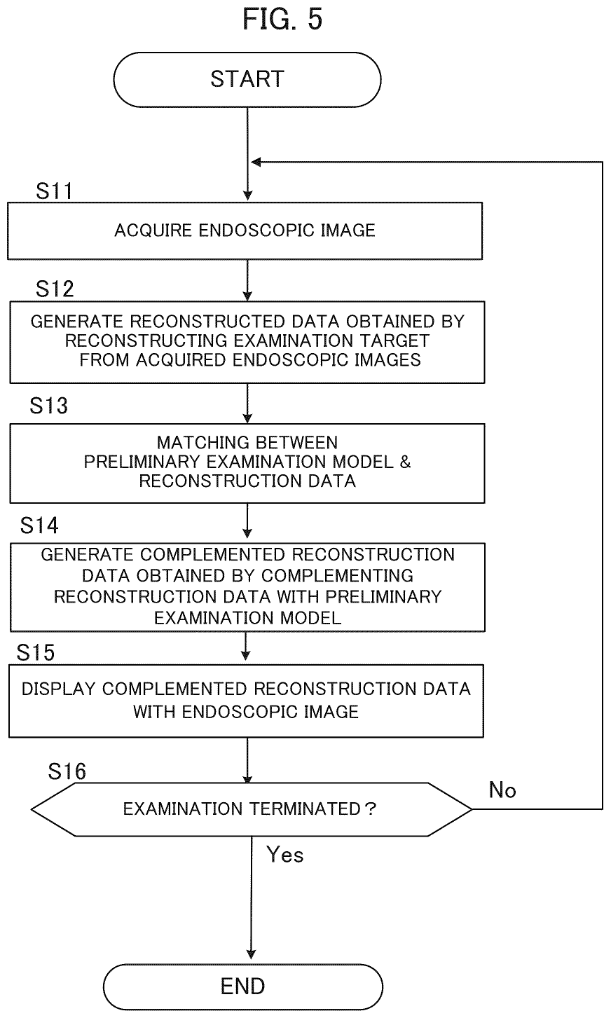

FIG. 5 is an example of a flowchart illustrating an outline of a display process that is executed by the image processing device 1 during the endoscopic examination in the first example embodiment. Here, as a typical example, complemented reconstruction data Mrc is generated and displayed during endoscopy using endoscopic images obtained during the endoscopic examination. It is noted that, instead of this embodiment, the image processing device 1 may generate and display the complemented reconstruction data Mrc after the endoscopic examination using the endoscope images obtained during the endoscopic examination.

First, the image processing device 1 acquires the endoscopic image Ic (step S11). In this instance, the endoscopic image acquisition unit 30 of the image processing device 1 receives the endoscopic image Ic from the endoscope 3 through the interface 13.

Next, the image processing device 1 generates reconstruction data Mr obtained by three-dimensionally reconstructing the examination target from a plurality of endoscopic images Ic acquired at step S11 (step S12). In this instance, the three-dimensional reconstruction unit 31 of the image processing device 1 generates the reconstruction data Mr using a technique such as the SfM on the basis of the endoscopic images Ic acquired during the period from the start of the examination to the current processing time.

Next, the image-processing device 1 performs matching between the preliminary examination model Mp and the reconstruction data Mr (step S13). In this instance, the matching unit 32 of the image processing device 1 generates the matching result by performing non-rigid registration between the preliminary examination model Mp acquired from the preliminary examination information storage unit 22 and the reconstruction data Mr generated by the three-dimensional reconstruction unit 31.

Then, based on the matching result, the image processing device 1 generates the complemented reconstruction data Mrc which is the reconstruction data Mr complemented with the preliminary examination model Mp (step S14). Then, the image processing device 1 displays the complemented reconstruction Mrc on the display device 2 together with the latest endoscopic image Ic (step S15).

Next, the image processing device 1 determines whether or not the endoscopic examination has been terminated (step S16). For example, upon detecting a predetermined input or the like from the input unit 14 or the operation unit 36, the image processing device 1 determines that the endoscopic examination has been terminated. Upon determining that the endoscopic examination has been terminated (step S16; Yes), the image processing device 1 terminates the process of the flowchart. On the other hand, upon determining that the endoscopic examination has not been terminated (step S16; No), the image processing device 1 gets back to the process at step S11. Then, the image processing device 1 acquires the endoscopic image Ic newly generated by the endoscope 3 at step S11, and includes the acquired endoscopic image Ic in the processing target, and re-executes the processes at step S12 to step S15.

A supplementary description will be given of the process of generating the preliminary examination model Mp to be stored in the preliminary examination data storage unit 22. Hereinafter, for convenience of explanation, it is assumed that the process will be executed by the image processing device 1, but the process may be executed by any device other than the image processing device 1. In the latter case, after the preliminary examination model Mp is generated by any device, the generated preliminary examination model Mp is stored in the memory 12 (the preliminary examination information storage unit 22 in detail) through data communication or a removable storage medium or the like.

First, the image processing device 1 acquires preliminary scan data such as a 3D-CT image or MRI data obtained by photographing the target organ of examination of the subject. Then, based on the user input, the image processing device 1 extracts the region of the target organ of examination from the preliminary scan data. In this case, for example, the image processing device 1 displays the preliminary scan data on the display device 2 and receives, from the input unit 14, a user input that specifies the region of the target organ of examination. Then, the image processing device 1 generates volume data, which represents the region of the target organ of examination, and which is extracted from the preliminary scan data of the subject. The volume data is, for example, three-dimensional voxel data representing the region of the target organ of examination in binary using 0 and 1. Next, the image processing device 1 generates three-dimensional preliminary examination model Mp that is a surface model from the above-described volume data. In this instance, the image processing device 1 converts the volume data into the preliminary examination model Mp using any algorithm for converting voxel data into polygon data. Examples of the above algorithm include the marching cubes method and the marching tetrahedra method. The generated preliminary examination model Mp is stored in the memory 12 (specifically, the preliminary examination information storage unit 22) which can be referred to by the image processing device 1.

Next, the matching process at step S13 will be supplemented.

First, the matching unit 32 extracts feature points as landmarks from the preliminary examination model Mp and the reconstruction data Mr, respectively. In this instance, the matching unit 32 applies a three-dimensional smoothing to the reconstruction data Mr, and then, based on the point clouds constituting the smoothed reconstruction data Mr and the connected graph thereof, the matching unit 32 extracts the feature points that become characteristic in the point clouds. In this instance, the matching part 32 performs the above-mentioned feature point extraction by using an arbitrary point group feature extraction technique, such as principal component analysis (PCA: Principal Component Analysis) of the point cloud and DoCoG (Difference of Center of Gravity), for example. In some embodiments, the preliminary examination model Mp may be provided with an identification label for each feature point to be extracted.

Next, the matching unit 32 determines the correspondence between the feature points extracted from the preliminary examination model Mp and the feature points extracted from the reconstruction data Mr, respectively, and then performs rigid body registration (alignment) between the preliminary examination model Mp and the reconstruction data Mr. In this instance, the matching unit 32 translates (includes rotate) at least one of the preliminary examination model Mp and/or the reconstruction data Mr so that the distances between corresponding feature points are minimized. Next, the matching unit 32 morphs the preliminary examination model Mp with reference to the reconstruction data Mr. In this instance, the matching unit 32 performs matching (alignment) between the preliminary examination model Mp and the reconstruction data Mr using a method of matching point clouds such as ICPD (Iterative Coherent Point Drift) and then moves any points other than the points that are regarded as the feature points (landmarks) in the preliminary examination model Mp.

### (5) Examiner Confirmation Screen Image

Next, each display example (first display example to fourth display example) of the examiner confirmation screen image displayed on the display device 2 by the display control unit 34 will be described in detail. The display control unit 34 generates display information Id for displaying the examiner confirmation screen image and supplies the display information Id to the display device 2, to thereby display the examiner confirmation screen image on the display device 2.

FIG. 6 illustrates a first display example of the examiner confirmation screen image. In the first display example, the display control unit 34 displays, on the examiner confirmation screen image, a complemented reconstruction image 41, which is an image obtained by projecting the complemented reconstruction data Mrc from a particular viewpoint (for example, the front direction of the subject) onto the two-dimensional plane, together with the latest endoscope image Ic generated by the endoscope scope 3.

In the first display example, the complement unit 33 generates the complemented reconstruction data Mrc obtained by complementing the reconstruction data Mr, which is generated based on the endoscopic images Ic obtained up to the current display time, with the preliminary examination model Mp corresponding to the non-photographed region, and the display control unit 34 displays the complemented reconstruction image 41 based on the complemented reconstruction data Mrc on the examiner confirmation screen image. It is noted that the display control unit 34 transparently displays an inner wall or the like on the viewpoint side in the complemented reconstruction image 41 so that the structure in the lumen of the target organ of examination can be visually recognized, for example.

Further, the complement unit 33 herein displays, on the complemented reconstruction image 41, the region (hatched region) of the target organ of examination based on the reconstruction data Mr and the region (non-hatched region) of the target organ of examination based on the preliminary examination model Mp in different display modes. Generally, since the endoscopic image Ic is a color image and the CT or MRI scan image obtained in the preliminary examination is a monochrome image, the reconstruction data Mr in the complemented reconstruction data Mrc includes color information (for example, RGB information) while the preliminary examination model Mp in the complemented reconstruction data Mrc does not include the color information. Accordingly, for example, when generating the complemented reconstruction image 41, the display control unit 34 displays, in color, the portion (i.e., portion corresponding to the reconstruction data Mr) which includes the color information, while displaying in monochrome, the portion (i.e., portion corresponding to the preliminary examination model Mp) which does not include the color information. Accordingly, the display control unit 34 can display, on the complemented reconstruction image 41, the photographed region (i.e., the portion corresponding to the generated reconstruction data Mr) and the non-photographed region (i.e., the portion not corresponding to the generated reconstruction data Mr) in each identifiable manner.

Furthermore, in the first display example, on the basis of the meta data stored in the preliminary examination information storage unit 22, the display control unit 34 recognizes the part of interest registered in the preliminary examination or the like and displays the preliminary examination mark 43 representing the part of interest on the corresponding part on the complemented reconstruction image 41. Furthermore, in the first display example, the display control unit 34 displays, over the complemented reconstruction image 41, an endoscope icon 42A schematically representing a part of the current endoscope 3 and a photographed region icon 42B schematically representing the photographed range of the endoscope 3. In this case, the display control unit 34 estimates the position and the photographed region of the endoscope 3 within the reconstruction data Mr and the complemented reconstruction data Mrc, based on the relative position and posture of the photographing unit of the endoscope 3 that are obtained as an execution result of the SfM performed in generating the reconstruction data Mr. Then, the display control unit 34 displays the endoscope icon 42A and the photographed region icon 42B based on the estimated result.

According to the first display example, the display control unit 34 can present a detailed overall image in three dimensions of the target organ of examination and the current photographing position in the target organ of examination and the position of the part of interest to the examiner.

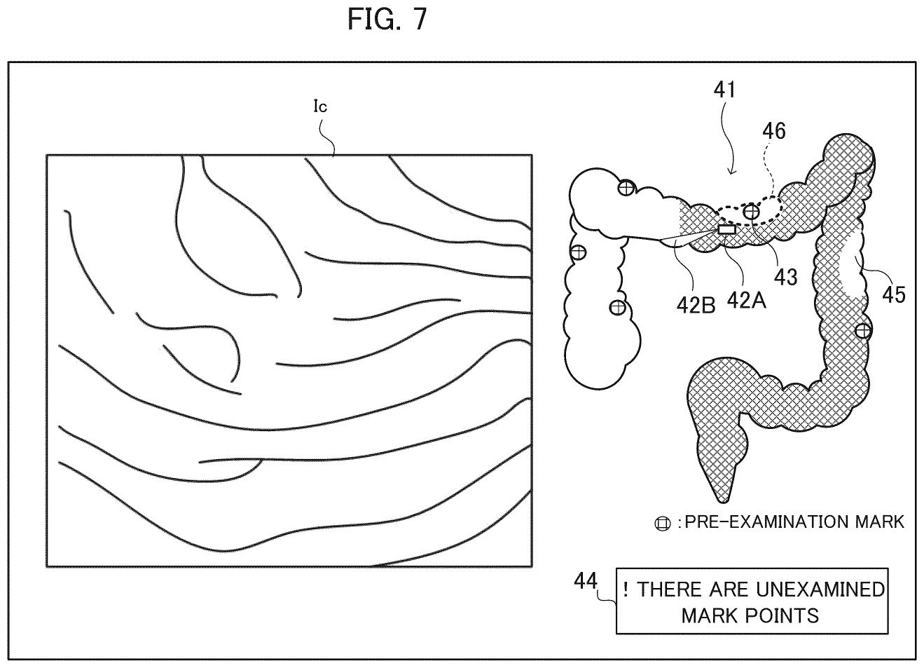

FIG. 7 shows a second display example of the examiner confirmation screen image. In the second display example, there are holes that have occurred in the reconstruction data Mr, and the display control unit 34 displays the complemented reconstruction image 41 representing the complemented reconstruction data Mrc in which the non-photographed regions corresponding to the holes are patched by the preliminary examination model Mp. Furthermore, in the second display example, the display control unit 34 displays information regarding the non-photographed regions corresponding to the holes.

Specifically, in the second display example, there are two holes in the reconstruction data Mr, and the first patched region 45 and the second patched region 46 where the portions corresponding to the two holes are patched by the preliminary examination model Mp are clearly indicated on the complemented reconstruction image 41. Here, the first patched region 45 and the second patched region 46 have a display mode different from the display mode of the region corresponding to the reconstruction data Mr since the former is a monochrome display and the latter is a color display.

Furthermore, in the second patched region 46, there are parts of interest based on the preliminary examination information, and the corresponding preliminary examination marks 43 are displayed on the second patched region 46. Therefore, in this case, the display control unit 34 considers the second patched region 46 as the non-examined region which the examiner has overlooked, and highlights and displays the second patched region 46 by the edging effect or the like (here, the addition of a broken line frame). Furthermore, the display control unit 34 displays a notification window 44 to the effect that there are un-examined points of interest. Thus, the display control unit 34 can suitably notify the examiner of the presence of the non-examined region which has been overlooked by the examiner.

Thus, according to the second display example, non-examined regions are detected based on the regions corresponding to the holes in the reconstruction data Mr, and the information regarding the non-examined regions is displayed, which encourages the examiner to look into the non-examined regions. Thus, the image processing device 1 can prevent the examiner from overlooking parts required to be examined and support the examiner to conduct the examination.

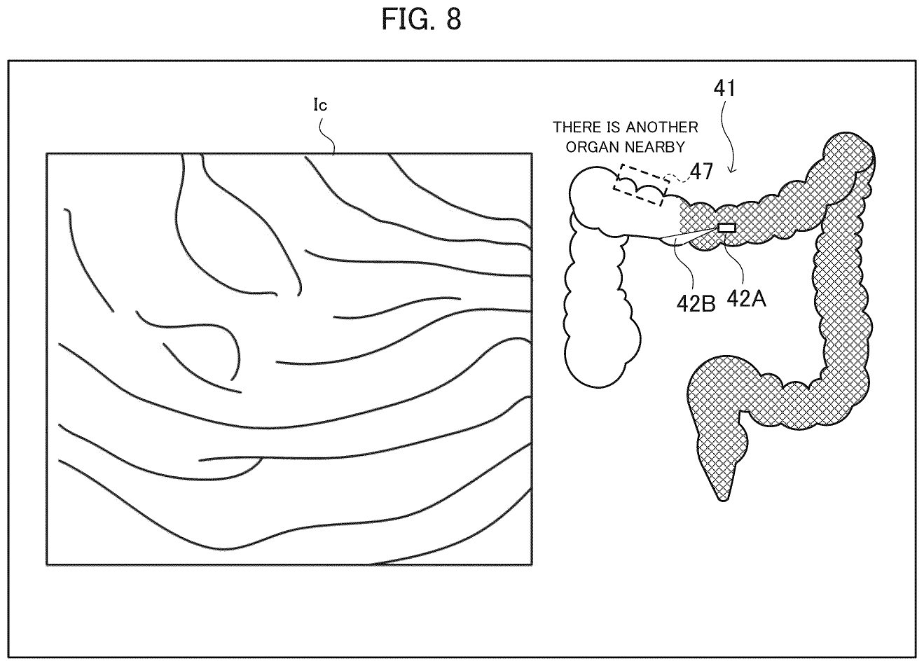

FIG. 8 illustrates a third display example of the examiner confirmation screen image. In the third display example, the display control unit 34 displays information regarding other organs present in the vicinity of the target organ of examination.

In this example, based on the information regarding other organ(s) present in the vicinity of the target organ of examination, the display control unit 34 highlights the region of the target organ of examination adjacent to the other organ(s) by surrounding the region in the dashed frame 47 on the complemented reconstruction image 41. Further, the display control unit 34 displays, in addition to the dashed frame 47, the text to the effect that there is another organ nearby. The information regarding the other organ(s) present in the vicinity of the target organ of examination is stored in advance in the memory 12 or the like. For example, in the meta data of the preliminary examination information stored in the preliminary examination information storage unit 22, information regarding a region of the target organ of examination adjacent to the other organ(s) is registered as a point of interest. In this case, by referring to this information, the display control unit 34 identifies and highlights the region of the target organ of examination adjacent to the other organ(s) on the complemented reconstruction image 41. This makes it possible to remind the examiner to recognize such a place where special attention is necessary in relation with other organs.

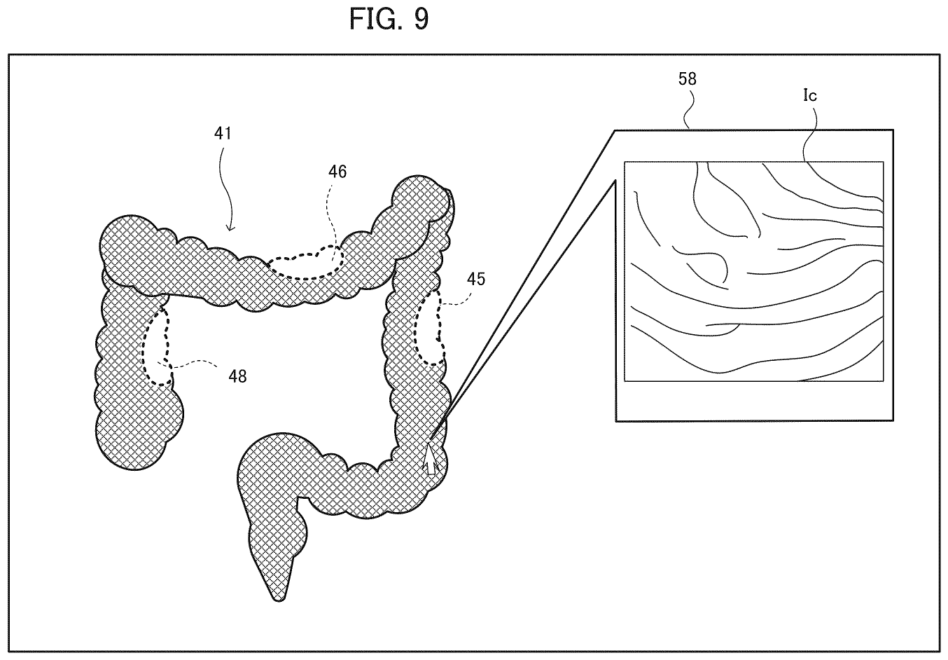

FIG. 9 shows a fourth display example of the examiner confirmation screen image. In the fourth display example, after the endoscopic examination is completed, the display control unit 34 displays the complemented reconstruction image 41 representing the complemented reconstruction data Mrc on the examiner confirmation screen image.

In this instance, the complement unit 33 generates complemented reconstruction data Mrc that is complemented by patching holes in the reconstruction data Mr, which are generated based on the endoscopic images Ic captured during endoscopic examination, using the preliminary examination model Mp. Then, the display control unit 34 displays the complemented reconstruction image 41 based on the complemented reconstruction data Mrc. In this case, based on the preliminary examination model Mp, the complement unit 33 generates the first patched region 45, the second patched region 46, and the third patched region 48 corresponding to the three holes in the reconstruction data Mr. Then, the display control unit 34 displays, on the complemented reconstruction image 41, these regions with a display mode different from the display mode of the region based on the reconstruction data Mr.

According to the fourth display example, the display control unit 34 receives an input for specifying an arbitrary position of the target organ of examination on the complemented reconstruction image 41 through the input unit 14 such as a mouse. In FIG. 9, the display control unit 34, as an example, displays a cursor and receives an input specifying the above-described position by the cursor. Then, the display control unit 34 displays the endoscopic image Ic corresponding to the specified position in association with the specified position. Here, the display control unit 34 displays the balloon 58 indicating the specified position, and displays the endoscopic image Ic in the balloon 58. In the case of the fourth display example, during the endoscopic examination, the image processing device 1 stores information, such as the photographing position of each endoscopic image Ic estimated in generating the reconstruction data Mr, in the endoscopic image storage unit 21 in association with the each endoscopic image Ic. Then, when performing the display based on the fourth display example, the display control unit 34 extracts the corresponding endoscopic image Ic from the endoscopic image storage unit 21 on the basis of the position on the complemented reconstruction data Mrc specified by the user and displays the extracted endoscopic image Ic on the balloon 58. Instead of displaying on the balloon 58 the endoscopic image Ic having the photographing position of the photographing unit corresponding to the position specified by the user, the display control unit 34 may display on the balloon 58 the endoscopic image Ic including the position specified by the user as a photographic subject.

In some embodiments, the display control unit 34 extracts preliminary scan data (CT image or MRI image) representing the position specified by the user from the preliminary examination information storage unit 22 and displays the preliminary scan data together with the endoscopic image Ic or instead of the endoscopic image Ic. For example, upon receiving the designation of the position of any one of the first patched region 45, the second patched region 46, and the third patched region 48 by the user, the display control unit 34 extracts the preliminary scan data corresponding to the position from the preliminary examination information storage unit 22 and displays the preliminary scan data on the balloon 58.

Thus, in the fourth display example, the image processing device 1 can present the whole target organ of examination of the subject at the confirmation stage or the like after the endoscopic examination.

### Second Example Embodiment

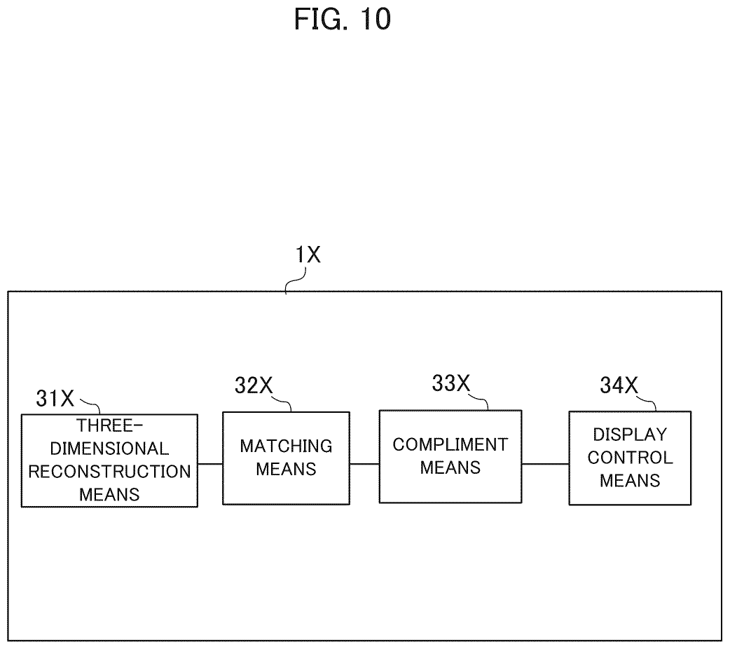

FIG. 10 is a block diagram of an image processing device 1X according to the second example embodiment. The image processing device 1X mainly includes a three-dimensional reconstruction means 31X, a matching means 32X, a complement means 33X, and a display control means 34X. The image processing device 1X may be configured by a plurality of devices.

The three-dimensional reconstruction means 31X is configured to generate, based on endoscopic images of an examination target captured by a photographing unit provided in an endoscope, reconstruction data obtained by three-dimensionally reconstructing the examination target. In this instance, the three-dimensional reconstruction means 31X may be configured to receive the endoscopic images directly from the photographing unit, or may be configured to acquire the endoscopic images from a storage device that stores the endoscopic images captured by the photographing unit. Examples of the “examination target” include a large bowel, a stomach, and other organs. Examples of the three-dimensional reconstruction means 31X include the three-dimensional reconstruction unit 31 in the first example embodiment.

The matching means 32X is configured to perform matching between a three-dimensional model of the examination target and the reconstruction data. The matching means 32X may be configured to acquire the three-dimensional model of the examination target from the memory of the image processing device 1X or may be configured to acquire the three-dimensional model from an external device separate from the image processing device 1X. Examples of the matching means 32X include the matching unit 32 according to the first example embodiment.

The complement means 33X is configured to generate, based on a result of the matching, complemented reconstruction data which is the reconstruction data complemented with the three-dimensional model. Examples of the complement means 33X include the complement unit 33 in the first example embodiment. The display control means 34X is configured to display the complemented reconstruction data on a display device. Examples of the display device include a display unit of the image processing device 1X and a device separate from the image processing device 1X. Examples of the display control means 34X include the display control unit 34 in the first example embodiment.

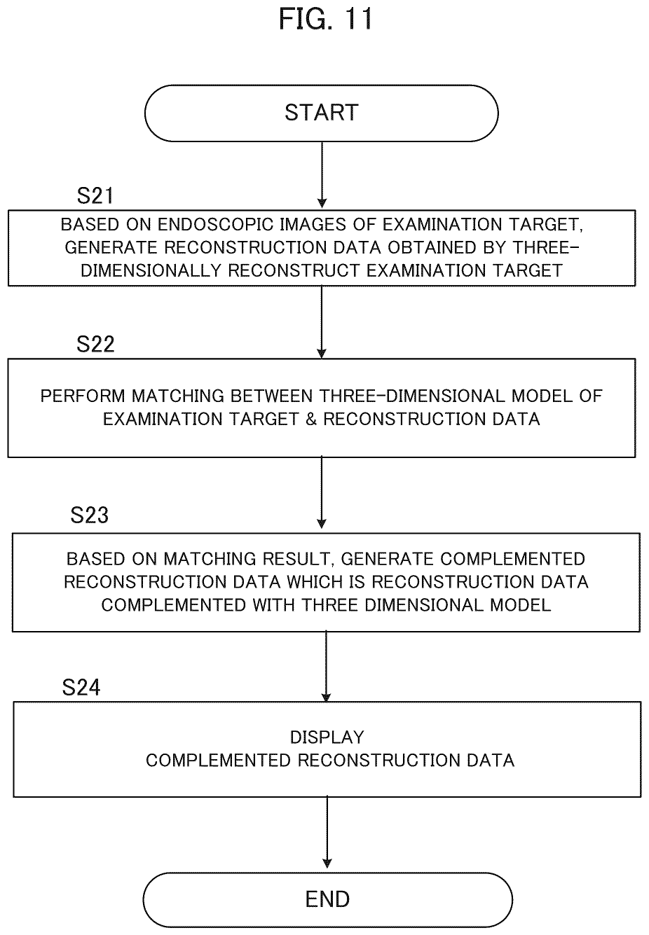

FIG. 11 is an exemplary flowchart of a processing procedure that is executed by the image processing device 1X in the second example embodiment. The three-dimensional reconstruction means 31X generates, based on endoscopic images of an examination target captured by a photographing unit provided in an endoscope, reconstruction data obtained by three-dimensionally reconstructing the examination target (step S21). The matching means 32X performs matching between a three-dimensional model of the examination target and the reconstruction data (step S22). The complement means 33X generates, based on a result of the matching, complemented reconstruction data which is the reconstruction data complemented with the three-dimensional model (step S23). The display control means 34X displays the complemented reconstruction data on a display device (step S24).

According to the second example embodiment, the image processing device 1X displays information regarding meta data preliminarily associated with the three-dimensional model of the examination target.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### Supplementary Note 1

An image processing device comprising:
a three-dimensional reconstruction means configured to generate, based on endoscopic images of an examination target captured by a photographing unit provided in an endoscope,
   reconstruction data obtained by three-dimensionally reconstructing the examination target;
a matching means configured to perform matching between a three-dimensional model of the examination target and the reconstruction data;
a complement means configured to generate, based on a result of the matching, complemented reconstruction data which is the reconstruction data complemented with the three-dimensional model; and
a display control means configured to display the complemented reconstruction data on a display device.

### Supplementary Note 2

The image processing device according to Supplementary Note 1,
wherein the display control means is configured to display the complemented reconstruction data on the display device in a state where a display mode of a region of the examination target based on the three-dimensional model is different from a display mode of a region of the examination target based on the reconstruction data.

[Supplementary Note 3] The image processing device according to Supplementary Note 1 or 2,
wherein the complement means is configured to generate the complemented reconstruction data obtained by adding the three-dimensional model corresponding to a region of the examination target which is not shown in the endoscopic images to the reconstruction data.

### Supplementary Note 4

The image processing device according to any one of Supplementary Notes 1 to 3,
wherein the complement means is configured to
   generate, based on the three-dimensional model, data for patching a hole in the reconstruction data of the examination target, and
   generate the complemented reconstruction data by adding the data to the reconstruction data.

### Supplementary Note 5

The image processing device according to Supplementary Note 4,
wherein the display control means is configured to display information regarding a non-examined region of the examination target based on the region of the examination target corresponding to the hole.

### Supplementary Note 6

The image processing device according to any one of Supplementary Notes 1 to 5,
wherein the display control means is configured to display, together with the complemented reconstruction data, information regarding another organ adjacent to the examination target on the display device.

### Supplementary Note 7

The image processing device according to Supplementary Note 6,
wherein the display control means is configured to highlight a region of the examination target adjacent to the other organ, on an image representing the complemented reconstruction data.

### Supplementary Note 8

The image processing device according to any one of Supplementary Notes 1 to 7,
wherein the three-dimensional model is data generated based on scan data of the examination target obtained in a preliminary examination conducted prior to an examination using the endoscope.

### Supplementary Note 9

The image processing device according to any one of Supplementary Notes 1 to 8,
wherein the display control means is configured to display a position and a photographed range of the endoscope, on an image representing the complemented reconstruction data.

### Supplementary Note 10

The image processing device according to any one of Supplementary Notes 1 to 9,
wherein the display control means is configured to display information indicating a part of interest to be noticed in the examination target, on an image representing the complemented reconstruction data.

### Supplementary Note 11

An image processing method executed by a computer, the image processing method comprising:
generating, based on endoscopic images of an examination target captured by a photographing unit provided in an endoscope,
   reconstruction data obtained by three-dimensionally reconstructing the examination target;
performing matching between a three-dimensional model of the examination target and the reconstruction data;
generating, based on a result of the matching, complemented reconstruction data which is the reconstruction data complemented with the three-dimensional model; and
displaying the complemented reconstruction data on a display device.

### Supplementary Note 12

A storage medium storing a program executed by a computer, the program causing the computer to:
generate, based on endoscopic images of an examination target captured by a photographing unit provided in an endoscope,
   reconstruction data obtained by three-dimensionally reconstructing the examination target;
perform matching between a three-dimensional model of the examination target and the reconstruction data;
generate, based on a result of the matching, complemented reconstruction data which is the reconstruction data complemented with the three-dimensional model; and
display the complemented reconstruction data on a display device.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1X Image processing device
2 Display device
3 Endoscope
11 Processor
12 Memory
13 Interface
14 Input unit
15 Light source unit
16 Audio output unit
100 Endoscopic examination system

What is claimed is:

1. An image processing device comprising:
at least one memory configured to store instructions; and
at least one processor configured to execute the instructions to:
generate, based on endoscopic images of an examination target captured by an endoscope,
reconstruction data obtained by three-dimensionally reconstructing the examination target;
perform matching between a three-dimensional model of the examination target and the reconstruction data;
generate, based on a result of the matching, complemented reconstruction data which is the reconstruction data complemented with the three-dimensional model; and
display the complemented reconstruction data on a display device.

2. The image processing device according to claim 1,
wherein the least one processor is configured to execute the instructions to display the complemented reconstruction data on the display device in a state where a display mode of a region of the examination target based on the three-dimensional model is different from a display mode of a region of the examination target based on the reconstruction data.

3. The image processing device according to claim 1,
wherein the least one processor is configured to execute the instructions to generate the complemented reconstruction data obtained by adding the three-dimensional model corresponding to a region of the examination target which is not shown in the endoscopic images to the reconstruction data.

4. The image processing device according to claim 1,
wherein the least one processor is configured to execute the instructions to
generate, based on the three-dimensional model, data for patching a hole in the reconstruction data of the examination target, and
generate the complemented reconstruction data by adding the data to the reconstruction data.

5. The image processing device according to claim 4,
wherein the least one processor is configured to execute the instructions to display information regarding a non-examined region of the examination target based on the region of the examination target corresponding to the hole.

6. The image processing device according to claim 1,
wherein the least one processor is configured to execute the instructions to display, together with the complemented reconstruction data, information regarding another organ adjacent to the examination target on the display device.

7. The image processing device according to claim 6,
wherein the least one processor is configured to execute the instructions to highlight a region of the examination target adjacent to the other organ, on an image representing the complemented reconstruction data.

8. The image processing device according to claim 1,
wherein the three-dimensional model is data generated based on scan data of the examination target obtained in a preliminary examination conducted prior to an examination using the endoscope.

9. The image processing device according to claim 1,
wherein the least one processor is configured to execute the instructions to display a position and a photographed range of the endoscope, on an image representing the complemented reconstruction data.

10. The image processing device according to claim 1,
wherein the least one processor is configured to execute the instructions to display information indicating a part of interest to be noticed in the examination target, on an image representing the complemented reconstruction data.

11. An image processing method executed by a computer, the image processing method comprising:
generating, based on endoscopic images of an examination target captured by an endoscope,
reconstruction data obtained by three-dimensionally reconstructing the examination target;
performing matching between a three-dimensional model of the examination target and the reconstruction data;
generating, based on a result of the matching, complemented reconstruction data which is the reconstruction data complemented with the three-dimensional model; and
displaying the complemented reconstruction data on a display device.

12. A non-transitory computer readable storage medium storing a program executed by a computer, the program causing the computer to:
generate, based on endoscopic images of an examination target captured by a an endoscope,
reconstruction data obtained by three-dimensionally reconstructing the examination target;
perform matching between a three-dimensional model of the examination target and the reconstruction data;
generate, based on a result of the matching, complemented reconstruction data which is the reconstruction data complemented with the three-dimensional model; and
display the complemented reconstruction data on a display device.
